(19) United States
(12) Patent Application Publication
(10) Pub. No.: US 2018/0038871 A1
(43) Pub. Date: Feb. 8, 2018

(54) DIAGNOSTIC BIOMARKER TO IDENTIFY WOMEN AT RISK FOR PRETERM DELIVERY

(71) Applicant: CEDARS-SINAI MEDICAL CENTER, Los Angeles, CA (US)

(72) Inventors: Ozlem EQUILS, Sherman Oaks, CA (US); Charles F. SIMMONS, JR., Los Angeles, CA (US)

(73) Assignee: CEDARS-SINAI MEDICAL CENTER, Los Angeles, CA (US)

(21) Appl. No.: 15/683,487

(22) Filed: Aug. 22, 2017

Related U.S. Application Data
(63) Continuation of application No. 14/836,013, filed on Aug. 26, 2015, now abandoned.
Application No. 14/836,013 is a continuation of application No. 13/512,212, filed on May 25, 2012, now abandoned.
(60) Provisional application No. 61/267,693, filed on Dec. 8, 2009.

(51) Int. Cl. G01N 33/68

(52) U.S. Cl. CPC G01N 33/689; G01N 2333/5428; G01N 2333/5437; G01N 2333/54; G01N 2800/368

(57) ABSTRACT

The invention relates to biomarkers associated with preterm delivery. More specifically, the invention provides methods of measuring biomarkers found in women that are at risk for preterm delivery.

### CROSS-REFERENCE TO RELATED APPLICATIONS

The present application is a continuation of U.S. application Ser. No. 14/836,013, filed Aug. 26, 2015, now abandoned, which is a continuation of U.S. application Ser. No. 13/512,212, now abandoned, which is a national stage application of International Application No. PCT/US2010/059248, filed Dec. 7, 2010, which claims the benefit of the filing date of U.S. Provisional Application No. 61/267,693 filed Dec. 8, 2009, the disclosure of each of which is incorporated herein by reference in its entirety.

### FIELD OF INVENTION

This invention relates to preterm delivery. More specifically, the invention provides biomarkers and methods of using biomarkers for determining preterm delivery risk.

### BACKGROUND

Preterm delivery is one of the most important fetal health problems in the United States today. Approximately one in eight newborns is delivered preterm and the incidence of prematurity has not decreased in the last 20 years. Most preterm babies, if they survive, often have cardiac, neurologic, ophthalmic, and gastrointestinal problems that can extend even beyond childhood, and perhaps lead to adult diseases such as atherosclerosis. Currently, there are few, if any diagnostic biomarkers available that effectively identify women who are going to deliver preterm. Biomarkers that are able to identify these women at risk would be useful in the deployment of prevention strategies. Thus, there is a need to develop novel diagnostics that may identify women who will deliver preterm. Especially, diagnostic biomarkers that may be detected in non-pregnant women or in women during the first trimester.

### SUMMARY OF THE INVENTION

In one embodiment, the invention includes methods of identifying a non-pregnant woman at risk for preterm delivery, comprising: obtaining a sample from the non-pregnant woman, determining the expression level of one or more biomarkers in the sample, and comparing the expression level of the one or more biomarkers with the expression level of biomarkers from those observed in women who delivered fullterm (controls). Since there is individual variation, the control values will constitute a range. If the level of expression of the one or more biomarkers in the sample is higher or lower than the level of expression of the same biomarkers in women who delivered fullterm (outside the range), then it is indicative that the non-pregnant woman is at risk for preterm delivery. Depending on how much the patient biomarker values are outside the range of the standard controls, the risk of preterm delivery may be determined as “low, medium or high.” The biomarkers include but not limited to IL-10, IL-13 and/or IL-1RA. The sample can comprise stimulated PBMC/whole blood supernatant (in the presence or absence of cortisol). Alternatively, the sample can comprise serum.

In another embodiment, the invention includes methods of diagnosing susceptibility of preterm delivery in a woman, comprising: obtaining a sample from the woman, determining the expression level of one or more diagnostic biomarkers in the sample, and comparing the expression level of the one or more biomarkers with the expression level of biomarkers from women who delivered fullterm (normal range). If the level of expression of the one or more diagnostic biomarkers in the sample is higher or lower than the level of expression of the same diagnostic biomarkers in the women who delivered fullterm (normal range), then it is indicative that the woman is at risk for preterm delivery. The diagnostic biomarkers can be inflammatory and/or anti-inflammatory cytokines. The diagnostic biomarkers selected include but not limited to IL-10, IL-13 and/or IL-1RA. Biomarker expression levels that are at least one to three times less than the biomarker expression levels of a control are indicative of preterm delivery. The sample can comprise stimulated PBMC/whole blood supernatant (in the presence or absence of cortisol). Alternatively, the sample can comprise serum. The women may be non-pregnant or in the first-trimester of pregnancy.

The present invention is also directed to a kit for preterm delivery. The kit is useful for practicing the inventive method of determining the risk of preterm delivery of non-pregnant women. The kit is an assemblage of materials or components, including a diagnostic bioassay of the present invention.

The exact nature of the components configured in the inventive kit depends on its intended purpose. For example, some embodiments are configured for the purpose of diagnosing susceptibility of women for preterm delivery. In one embodiment, the kit is configured particularly for the purpose of determining the risk of preterm delivery of non-pregnant women. In another embodiment, the kit is configured particularly for the purpose of diagnosing susceptibility of women for preterm delivery of pregnant or non-pregnant women.

Instructions for use may be included in the kit. “Instructions for use” typically include a tangible expression describing the technique to be employed in using the components of the kit to effect a desired outcome, such as to treat ischemia. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing a polyphenol analog. The packaging material generally has an external label which indicates the contents and/or purpose of the kit and/or its components.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1D: Graphical representation of expression levels of various biomarkers of preterm individuals as compared to full term individuals. Empirical means and standard errors for each cytokine and treatment group are presented. FIG. 1A shows the expression level of IL-2. FIG. 1B shows the expression level of IL-10. FIG. 1C shows the expression level of IFN-g. FIG. 1D shows the expression level of GMCSF.

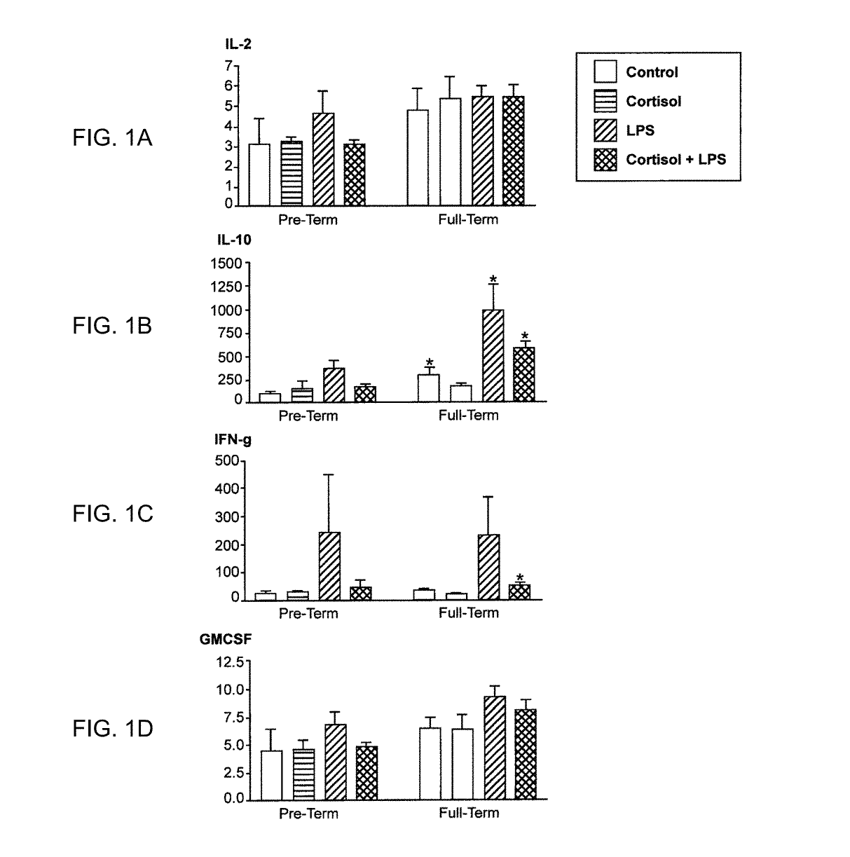

FIG. 2A-2D: Graphical representation of expression levels of various biomarkers of preterm individuals as compared to full term individuals. Empirical means and standard errors for each cytokine and treatment group are presented. FIG. 2A shows the expression level of IL-4. FIG. 2B shows the expression of IL-13. FIG. 2C shows the expression level of TNF-a. FIG. 2D shows the expression level of IL-IRA.

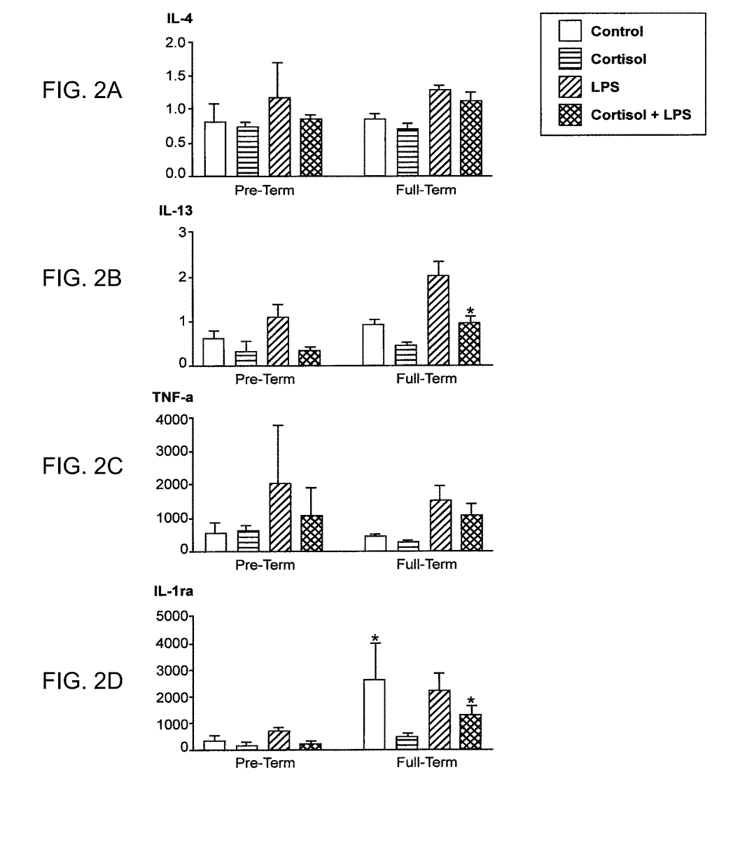

### DESCRIPTION OF THE INVENTION

Multiple factors lead to preterm delivery; however immune activation is thought be the final affector pathway that leads to preterm rupture of membranes and contractions. Family and genetic studies suggest that prematurity runs in families and history of prior preterm delivery increases the risk of future preterm deliveries. The inventors believed that these observations can be explained by the inherent differences in the immune responses of women who deliver preterm compared with those who deliver full term, and that these differences can be detected even in the non-pregnant state. The inventors believed that by detecting these differences one may be able to identify those women who are at risk to deliver preterm even before they become pregnant.

The inventors are the first to have discovered a biomarker assay that provides physicians with a tool to identify women who are at risk for preterm delivery, even before they become pregnant or while in the first trimester. Identification of women at risk allows the physician to better focus on preventative strategies in these women and improve pregnancy outcome. In one embodiment, the test may be done on small amounts of blood sample obtained from the subject, thus being minimally invasive to the fetus. Furthermore, the test may be repeated multiple times during the course of pregnancy. Thus, it provides dynamic assessment of the preterm delivery risk under the influence of changing environmental/physiologic factors, as wells as requires minimal skills to draw blood as opposed to obtaining amniotic fluid.

One skilled in the art will recognize many methods and materials similar or equivalent to those described herein, which could be used in the practice of the present invention. The present invention is in no way limited to the methods and materials described. For purposes of the present invention, the following terms are defined below.

As used herein, the term “preterm delivery” refers to a premature birth or conditions associated with a premature birth, including for example, a child delivered before 34 weeks of amenorrhea.

“Biomarker,” “diagnostic biomarker,” or “preterm delivery biomarker” refers to a molecular indicator that is associated with a particular pathological or physiological state. The “biomarker” as used herein is an indicator for preterm delivery. The indicator can be a cytokine or an immunomodulating agent, including interleukins and interferons. Examples of “biomarkers” include but are not limited IL-2, IL-4, IL-5, IL-8, IL-10, IL-12, IL-13, GM-CSF, IFN-g, and TNF-a. Preferably, biomarkers of the present invention include IL-10, IL-13 and/or IL-1RA. A “biomarker” of the present invention may be detected in a sample.

“Sample” or “a biological sample” refers to cells or component parts, or a fraction or portion thereof of body fluids, including but not limited to blood or amniotic cord blood. A “sample” or “biological sample” further refers to plasma, serum, and/or peripheral blood mononuclear cells (PMBC).

In one embodiment, the present invention provides a method of diagnosing susceptibility for preterm delivery in a non-pregnant woman comprising obtaining a sample from the woman and assaying the sample for the presence or absence of one or more diagnostic biomarkers, where the presence or absence of one or more diagnostic biomarkers is indicative of susceptibility for preterm delivery in the woman. In another embodiment, the sample comprises PMBC supernatant. In another embodiment, the sample comprises whole blood. In another embodiment, the sample comprises serum. In another embodiment, the one or more diagnostic biomarkers comprise a microbial component lipopolysacharide (LPS) or another immune stimulant induced cytokine expression profile. In another embodiment, the one or more diagnostic biomarkers comprise inflammation and/or anti-inflammatory cytokines. In another embodiment, the diagnostic biomarkers are analyzed in the presence of cortisol. In another embodiment, the cortisol concentration is in the range of 1 ug/ml to 500 ug/ml, with a preferred range of 1 ug/ml to 150 ug/ml. In another embodiment, the one or more diagnostic biomarkers comprise a low expression of IL-10, IL-13 and/or IL-1RA. In another embodiment, the low expression of IL-10, IL-13 and/or IL-1RA comprises a 1 to 3 fold decrease in expression compared to levels ordinarily found in a healthy individual.

There are many techniques readily available in the field for detecting the presence or absence of cytokines or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any numbers of techniques that may be employed to isolate and/or fractionate biomarkers. For example, a cytokine may be captured using biospecific capture reagents, such as antibodies, aptamers or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as cytokines may be detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, cytokines may also be fractionated to isolate them from other components in a solution or of blood that may interfere with detection. Fractionation may include platelet isolation from other blood components, sub-cellular fractionation of platelet components and/or fractionation of the desired cytokine from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Alternatively, for example, the presence of biomarkers such as cytokines may be detected using PCR techniques or flow cytometry. All of the available techniques for cytokine and chemokine detection may be used.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Biomarker Assay

Peripheral blood mononuclear cells (PBMC) from non-pregnant (at least 5-6 years post-partum) women with history of preterm or full term delivery, in addition to the microbial component lipopolysaccharide (LPS)-induced cytokine expression profile were examined. PBMC were separated from whole blood using Ficoll gradient. The cells were counted and equal numbers of cells were plated in 24 well plates. PBMC were treated with cortisol (50 or 300 ug/ml) or media for 1 hour prior to stimulation with LPS (0, 1, or 100 ug/ml) for 24 hours.

The PBMC were lysed and the supernatant was examined for inflammatory and anti-inflammatory cytokine expression by using Bioplex technology (Bio-rad). The inventors found that during the non-pregnant state IL10, IL13 and IL1Ra expression was lower in the PBMC obtained from women whom had previous preterm delivery, and that those biomarkers may be measured to identify women who were at risk for preterm delivery in the future.

In this study two patients were preterm and four were full term. There were 9 samples per patient (3×3 design) and all samples from each patient were analyzed twice for cytokine concentrations with the exception of one full term subject who only had one analysis of each sample. As a result there are a total of 99 observations for each cytokine measured. The assay simultaneously measured concentrations of 11 inflammatory markers: IL-1ra (IL1-receptor antagonist), IL-2, IL-4, IL-5, IL-8, IL-10, IL-12, IL-13, GM-CSF, IFN-g, and TNF-a.

### Example 2

### Establishment of Cut Off Points

Many samples were above or below the detection for some of the endpoints. Cut off points were established for each cytokine measured (Table 1). If a sample was out of the range of detection, a default value was assigned to the sample as indicated below.

TABLE 1
The establishment of cut off points for out of range values.

| | | | Fraction of |
|---|---|---|---|
| | | Out of Range | Samples out of |
| Cytokine | Limit of Detection | Set Value | Detection Range |
| IL-1ra | all samples within levels | n/a | 0/99 |
| | of detection | | |
| IL-2 | LL = 0.50 | 0.25 | 10/99 |
| IL-4 | LL = 0.14 | 0.08 | 6/99 |
| IL-5 | LL = 0.90 | 0.5 | 82/99 |
| IL-8 | UL = 95,000 | 100,000 | 79/99 |
| IL-10 | all samples within levels | n/a | 0/99 |
| | of detection | | |
| IL-12 | LL = 0.33 | 0.15 | 19/99 |
| IL-13 | LL = 0.17 | 0.08 | 8/99 |
| GM-CSF | LL = 1.18 | 0.60 | 11/99 |
| IFN-g | LL = 1.21 | 0.60 | 5/9 |
| TNF-a | all samples within levels | n/a | 0/99 |
| | of detection | | |

LL = Lower Limit;
UL = Upper Limit

All samples that were below the range of detection for IL-2, IL-4, IL-13, and IFN-g were from the two subjects with a history of pre-term delivery. Of the 11 samples assayed that were below detection limits for GM-CSF, only 1 was from a subject with a full-term delivery while the rest were from subjects with histories of pre-term deliveries.

### Example 3

### Statistical Analysis

Empirical means and standard errors for each cytokine and treatment group are presented herein. Here, the averages of replicate measures were calculated first for each subject. The mean and SEM was then taken across each treatment group.

All raw data was tested via the Kolmogorov-Smirnov test to determine if the data followed a normal distribution. Log-transformations were performed for all data found to have a non-normal distribution prior to further statistical analysis. For each cytokine, a mixed effects model was used to examine all data for significant effects with the outcome variable as the cytokine concentration; the fixed predictor variables as LPS concentration, cortisol concentration, and delivery status (pre- or full-term); and the random effect due to the replicate data points. To test the interaction of the three fixed predictor variables on cytokine concentration, both first and second level interactions ware added to each cytokine model. Post-hoc testing was performed using a Student's t-test.

### Example 4

### Cortisol and LPS Concentrations

All 99 observations were initially used in the analysis to first determine which concentrations of LPS and cortisol induced the most robust effect. For all cytokines measured there was no significant difference between the effects of 1 ug/ml and 100 ug/ml LPS. For several cytokines (IL-1ra, IL-10, and IL-13), 300 ug/ml cortisol had a more significant effect to suppress cytokine levels than 50 ug/ml.

As a result of the findings, the inventors focused on the data with 0 or 300 ug/ml of cortisol pretreatment crossed with 0 or 1 ug/ml LPS. This results in 4 samples per subject, each analyzed twice (with exception of the samples from the full term subject who only had one analysis of each sample). With 8 measurements per subject (with the exception of the full term subject who only has 4 measurements) this results in a data set with 44 measurements for each of the cytokines.

### Example 5

### Analysis of Testing for Cytokine Differences Between Groups

Using the same mixed model regression, estimated group means and standard errors were calculated. Differences between preterm and full-term pregnancies were considered significant where p <0.10.

Based on the results of the statistical modeling, cortisol did suppress secretion of IL-13 and IL-1ra; LPS increased secretion of IL-4, IL-10, IL-13, TNF-a, and IL-1ra; and women who had preterm deliveries had overall lower secretion levels of IL-10, IL-13, and IL-1ra (Table 2).

TABLE 2
Global effects of cortisol, LPS, and premature delivery on cytokine
secretion levels in PBMC based on results of mixed modeling.

| Cytokine | Cortisol Effect | LPS Effect | Pre-term Effect |
|---|---|---|---|
| IL-2 | n/c, p = .80 | n/c, p = .50 | n/c, p = .17 |
| IL-4 | n/c, p = .26 | increase, p = .07 | n/c, p = .59 |
| IL-10 | n/c, p = .12 | increase, p = .01 | decrease, p = .01 |
| IL-13 | decrease, p = .01 | increase, p = .04 | decrease, p = .02 |
| IFN-g | n/c, p = .10 | n/c, p = .17 | n/c, p = .22 |
| TNF-a | n/c, p = .41 | increase, p = .04 | n/c, p = .75 |
| GM-CSF | n/c, p = .51 | n/c, p = .19 | n/c, p = .17 |
| IL-1ra | decrease, p = .03 | increase, p = .09 | decrease, p = .01 |

n/c = No Change

Women who had pre-term deliveries had lower baseline IL-10, 1L13 and IL-1Ra expression compared to those who delivered full term. There were no differences in cytokine production between LPS-stimulated PBMC from preterm delivering women when compared with that from women with full-term deliveries. In other words, adjusting for unstimulated secretion levels, the concentration of cytokines released from LPS stimulation was no different between the two groups of women. In the presence of cortisol PBMC from women who had pre-term deliveries produced lower IL-13 and IFN-g, expression when compared with that from women with full-term deliveries.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

Many modifications and variations of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

Hutzal C E, Boyle E M, Kenyon S L, Nash J V, Winsor 5, Taylor D J, Kirpalani H. Use of antibiotics for the treatment of preterm parturition and prevention of neonatal morbidity: a metaanalysis. Am J Obstet Gynecol. 2008 December; I 99 (6):620.e1-8. Epub 2008 Oct 30.

Swamy G K, Ostbye T, Skjaerven R. Association of preterm birth with long-term survival, reproduction, and next-generation preterm birth. JAMA. 2008 Mar 26; 299 (12):1429-36. Erratum in: JAMA. 2008 Jul 9; 300 (2):170-1.

DeFranco E, Teramo K, Muglia L. Genetic influences on preterm birth. Semin Reprod Med. 2007 January; 25 (1):40-51.

Plunkett J, Muglia L J. Genetic contributions to preterm birth: implications from epidemiological and genetic association studies. Ann Med. 2008; 40(3):167-95.

Goldenberg R L, Culhane I F, lams J D, Romero R. Epidemiology and causes of preterm birth. Lancet. 2008 Jan. 5; 371 (9606):75-84.

Blank V, Hirsch E, Challis J R, Romero R, Lye Si. Cytokine signaling, inflammation, innate immunity and preterm labour—a workshop report. Placenta. 2008 March; 29 Suppl A:S102-4.

Mazaki-Tovi S, Romero R, Kusanovic J P, Erez 0, Pineles B L, Gotsch F, Mittal P, Than N G, Espinoza J, Hassan S S. Recurrent preterm birth. Semin Perinatol. 2007 June; 31 (3):142-58.

Romero R, Espinoza J, Goncalves L F, Kusanovic J P, Friel L, Hassan S. The role of inflammation and infection in preterm birth. Semin Reprod Med. 2007 January; 25 (1):21-39.

Romero R, Espinoza J, Gonsalves L F, Kusanovic J P, Friel L A, Nien J K. Inflammation in preterm and term labour and delivery. Semin Fetal Neonatal Med. 2006 October; 11 (5):317-26.

Luu T M, Ment L R, Schneider K C, Katz K H, Allan W C, Vohr B R. Lasting effects of preterm birth and neonatal brain hemorrhage at 12 years of age. Pediatrics. 2009 March; 123 (3):1037-44.—Morse S B, Zheng H, Tang Y, Roth J. Early school-age outcomes of late preterm infants. Pediatrics. 2009 April; 123 (4):e622-9.

Kamholz K L, Cole C H, Gray J E, Zupancic J A. Cost-effectiveness of early treatment for retinopathy of prematurity. Pediatrics. 2009 January; 123 (1):262-9.

Limperopoulos C, Bassan }I, Sullivan N R, Soul J S, Robertson R L Jr, Moore M, Ringer S A, Volpe J J, du Plessis A J. Positive screening for autism in ex-preterm infants: prevalence and risk factors. Pediatrics, 2008 April; 121 (4):758-65.

Jacobs S E, O'Brien K, Inwood 5, Kelly E N, Whyte H E. Outcome of infants 23-26 weeks of gestation pre and post surfactant. Acta Paediatr 2000, 89 (8):959-965.

Hack M, Flannery D J, Schluchter M, et al. Outcomes in young adulthood for very-low-birth-weight infants. N EngI Med 2002, 346 (3):149-157.

Gent, M R, Gerber S, Nesin M, Witkin S S. Polymorphism in the interleukin-1 gene complex and spontaneous preterm delivery. Am J Obstet Gynecol. 2002 July; 187 (1):157-63.

Peltier M R. Immunology of term and preterm labor. Reprod Biol Endocrinol. 2003 Dec. 2; 1:122.

Kalish R B, Vardhana S, Gupta M, Perni S C, Witkin S S, Interleukin-4 and -10 gene polymorphisms and spontaneous preterm birth in multifetal gestations. Am 1 Obstet Gynecol. 2004 March; 190 (3):702-6.

Gotsch F, Romero R, Kusanovic J P, Erex 0, Espinoza J, Kim C J, Vaisbuch E, Than N G, Mazaki-Tovi S Chaiworapongsa T, Mazor M, Yoon B H, Edwin S, Gomez R, Mittal P, Hassan S S, Sharma S. The antiinflammatory limb of the immune response in preterm labor, intra-amniotic infection/inflammation, and spontaneous parturition at term: a role for interleukin-10. J Matern Fetal Neonatal Med. 2008 August; 2 I (8):529-47.

Berghella V, Hayes E, Visintine J, Baxter J K. Fetal fibronectin testing for reducing the risk of preterm birth. Cochrane Database Syst Rev. 2008 Oct. 8; (4):CD006843.

Herbst A, Nilsson C. Diagnosis of early preterm labour. BJOG. 2006 December;113 Suppl 3:60-7. Review. Erratum in: BJOG. 2008 April; 115 (5):674-5.

Lockwood C J, Senyei A E, Dische M R. Casal D, Shah K D, Thung S N, Jones L. Deligdisch L, Garite D. Fetal fibronectin in cervical and vaginal secretions as a predictor of preterm delivery. N Engl J Med. 1991 Sep 5; 325 (10):669-74.

Honest H, Bachmann L M, Gupta J K, Kleijnen. 1, Khan K S. Accuracy of cervicovaginal fetal fibronectin test in predicting risk of spontaneous preterm birth: systematic review. BMJ. 2002 Aug. 10 ;325 (7359):301.

Most 0, Langer 0, Kerner R, David G B, Calderon!. Can myometrial electrical activity identify patients in preterm labor? Am J Obstet Gynecol. 2008 October; 199 (4):378.e1-6.

Menon R. Camargo M C, Thorsen P, Lombardi S J, Fortunate Si. Amniotic fluid interleukin-6 increase is an indicator of spontaneous preterm birth in white but not black Americans. Am. 1 Obstet Gynecol. 2008 January; 198 (1):77,e1-7.

Vogel 1, Goepfert A R, Thorsen P, Skogstrand K, Hougaard D M, Curry A H, Cliver S, Andrews W W. Early second-trimester inflammatory markers and short cervical length and the risk of recurrent preterm birth. J Reprod immune!. 2007 October; 75 (2):133-40. Epub 2007 Apr 17.

Kim KW, Romero R, Park H S, Park C W, Shim S S, Jun J K, Yoon B H. A rapid matrix metalloproteinase-8 bedside test for the detection of intraamniotic inflammation in women with preterm premature rupture of membranes. Am .1 Obstet Gynecol. 2007 September; 197 (3):292.el -5.

Spong C Y. Prediction and prevention of recurrent spontaneous preterm birth. Obstet Gynecol. 2007 August; 110 (2 Pt 1):405-15. Review.

Ghosh G. Breborowicz A, Brazen M, Maczkiewicz M, Kobelski M, Dubiel M, Gudmundsson S. Evaluation of third trimester uterine artery flow velocity indices in relationship to perinatal complications.1 Matern Fetal Neonatal Med. 2006 September; 19 (9):551-5.

Leitich H. Secondary predictors of preterm labour. BJOG. 2005 Mail] 12 Suppl 1:48-50. Review.

Challis J R. Maternal corticotropin-releasing hormone, fetal growth, and preterm birth. Am J Obstet Gynecol. 2004 October; 191 (4):1059-60.

Heine R P, McGregor J A, Goodwin T M, Anal R, Hayashi R H, Robertson P A, Varner M W. Serial salivary estriol to detect an increased risk of preterm birth. Obstet Gynecol. 2000 October; 96 (4):490-DWI 13662752v1 0067789-254 PRO

What is claimed is:

1. A method of identifying a non-pregnant woman at risk for preterm delivery, comprising:
(a) obtaining a sample from the non-pregnant woman;
(b) determining the expression level of one or more biomarkers in the sample;
(c) comparing the expression level of the one or more biomarkers with the expression level of biomarkers from women who delivered fullterm; and
(d) determining that the level of expression of at least one of each of the one or more biomarkers in the sample is higher or lower than the level of expression of the same biomarkers in the women who delivered fullterm.

2. The method according to claim 1, wherein the one or more biomarkers are IL-10, IL-13 and/or IL-1RA.

3. The method according to claim 1, wherein the expression levels of each of the one or more biomarkers in the sample are at least one to three times less than the expression levels of the same one or more biomarkers in the women who delivered fullterm.

4. The method according to claim 1, wherein the amount of the differences of the expression levels of the one or more biomarkers in the sample and the expression levels of the same one or more biomarkers from the women who delivered fullterm is indicative of low, medium or high risk of preterm delivery.

5. The method of claim 1, wherein the sample comprises PBMC or whole blood supernatant with or without cortisol treatment.

6. The method of claim 1, wherein the sample comprises serum.

7. A method of diagnosing susceptibility to preterm delivery in a woman, comprising:
(a) obtaining a sample from the woman;
(b) determining the expression level of one or more diagnostic biomarkers in the sample;
(c) comparing the expression level of each of the one or more diagnostic biomarkers in the sample with the expression level of each of one or more of the same biomarkers from women who delivered fullterm; and
(d) determining that the level of expression of at least one of each of the one or more diagnostic biomarkers in the sample is higher or lower than the level of expression of the same one or more diagnostic biomarkers in the women who delivered fullterm.

8. The method according to claim 7, wherein the one or more diagnostic biomarkers comprise inflammatory and/or anti-inflammatory cytokines.

9. The method according to claim 7, wherein the one or more diagnostic biomarkers are IL-10, IL-13 and/or IL-1RA.

10. The method according to claim 7, wherein the expression levels of the one or more diagnostic biomarkers in the sample are each at least one to three times less than the diagnostic biomarker expression levels of the control.

11. The method according to claim 7, wherein the amount of the differences of the expression levels of the one or more diagnostic biomarkers in the same sample and the expression levels of the same one or more diagnostic biomarkers from the women who delivered fullterm is indicative of low, medium or high susceptibility of preterm delivery.

12. The method of claim 7, wherein the sample comprises PBMC or whole blood supernatant with or without cortisol treatment.

13. The method of claim 7, wherein the sample comprises serum.

14. The method of claim 7, wherein the woman is not pregnant.

15. The method of claim 7, wherein the woman is in the first trimester of pregnancy.

16. A kit, comprising:
a biomarker assay; and
instructions for the use of the assay for identifying a non-pregnant woman at risk for preterm delivery.

17. The kit according to claim 16, wherein the wherein the biomarker is IL-10, IL-13 and/or IL-1RA.

18. A kit, comprising:
a diagnostic assay; and
instructions for the use of the assay for diagnosing susceptibility of a woman to preterm delivery.

19. The kit according to claim 18, wherein the diagnostic biomarker is IL-10, IL-13 and/or IL-1RA.
